(19) United States
(12) Patent Application Publication
(10) Pub. No.: US 2020/0146880 A1
(43) Pub. Date: May 14, 2020

(54) Portable Limb, Head, Chest Cooling System

(71) Applicant: Joseph Jones, Bowie, MD (US)

(72) Inventor: Joseph Jones, Bowie, MD (US)

(21) Appl. No.: 16/683,941

(22) Filed: Nov. 14, 2019

Related U.S. Application Data
(60) Provisional application No. 62/767,006, filed on Nov. 14, 2018.

(51) Int. Cl. A61F 7/02; A61F 7/10

(52) U.S. Cl. CPC A61F 7/02; A61F 2007/0056; A61F 7/10

(57) ABSTRACT

A personal portable cooling system and method which includes a device which rapidly cools the joints, extremities, chest, or head of a given patient and a coolant, such as a can of compressed gas/liquid is disclosed.

### CROSS REFERENCE

This application claims the benefit of the filing date of U.S. Provisional Patent Application Ser. No. 62/767,000, filed Nov. 14, 2018, which is hereby incorporated by reference in its entirety.

### FIELD

The present disclosure relates to a personal portable cooling system and method, and in particular to a personal portable cooling system and method that utilizes expansion of a coolant that travels through a tube to absorb energy and cool the adjacent environment.

### BACKGROUND

The treatment of heat injuries and acute extremity injuries have made utilization of cryo-cuffs and variations of the ‘ice sheets’ or cold blankets common practice in modern medicine. A brief search of Amazon.com reveals a host of devices which utilize cold to alleviate the pain associated with surgery or acute injury to an extremity. There are also several devices being sold by North American Rescue or similar companies which are created to rapidly reduce the core body temperature of a patient suffering from hyperthermia.

Of the most effective devices one can purchase, they all have the same thing in common, they require a freezer or similar device to create ice or to re-freeze ice packs. During a recent trip to the country of Georgia, we ran into issues as we were preparing for the possibility of a heat casualty while having no access to refrigerators, freezers, or ice packs.

There is a need for cooling devices which are both portable, and does not rely on the need for pre-formed ice cubes, or frozen ice packs. This would be useful to an Army Physician in the field, as well as patients who suffer from chronic arthritis in one or more joints.

The art lacks personal cooling devices that consist of a small, portable, closed system which is very similar to that of the modern refrigerator and does not require that medical personnel carry ice, cold water, or any previously cooled or frozen items to be effective.

### SUMMARY

In accordance with one aspect of the present disclosure, there is provided a method for cooling a selected portion of a person with a portable cooling system, including:
placing on a selected portion of a person a tubing system including an entry port, a tubing loop, a tubing exhaust port and a plurality of temperature sensors in fluid communication with a receiver;
securing a flow of coolant from a coolant container to the receiver;
closing an exit valve of the tubing exhaust port;
opening an entry valve of the tubing entry port; and
controlling the distribution of the coolant in the tubing system with a regulator, including a computer in communication with the entry valve, exit valve and the plurality of temperature sensors. In an embodiment, the method further includes opening the exit valve to flush the system with the coolant present in the tubing system, closing the exit valve and opening the entry valve allowing coolant into the tubing system.

In accordance with another aspect of the present disclosure, there is provided a personal portable cooling system, including:
a receiver including a molded entry port adapted to receive and secure a flow of coolant from a coolant container;
a tubing system including an entry port, a tubing loop, a tubing exhaust port and a plurality of temperature sensors in fluid communication with the receiver;
an entry valve disposed in the tubing entry port;
an exit valve disposed in the tubing exhaust port; and
a regulator, including a computer in communication with the entry valve, exit valve and the plurality of temperature sensors which controls the distribution of the coolant in the tubing system.

These and other aspects of the present disclosure will become apparent upon a review of the following detailed description and the claims appended thereto.

### BRIEF DESCRIPTION OF THE DRAWINGS

FIG. 1 is a schematic drawing of an embodiment of the system;

FIG. 2 is a schematic drawing of an embodiment of a knee pad of the system in place;

FIG. 3 shows the control interface of the overall structure of an embodiment of the device;

FIG. 4 shows the attachments of the overall structure of an embodiment of the device;

FIG. 5 shows a compressed air can which plugs into the receiver and connected by tubing to a knee pad;

FIG. 6 shows the exhaust vent which allows expanded liquid to release from the system;

FIG. 7 shows a receiver interfacing with a compressed air can;

FIG. 8 shows expanding liquid and gas leaving system through exhaust vent;

FIG. 9 shows a belt attachment;

FIG. 10 shows a clip device;

FIG. 11 shows a vest configuration; and

FIG. 12 shows a knee pad configuration.

### DETAILED DESCRIPTION

The present disclosure relates to a personal portable cooling system which includes a device which rapidly cools the joints, extremities, chest, or head of a given patient and a coolant. A suitable coolant includes a liquid that cools as it expands to a gas, such as a gas compressed to liquid form, or a gas that cools as it expands, such as a compressed gas. A suitable gas includes CO₂ that cools as it expands and compressed air. Specifically, a suitable liquid includes for example, diflouroethane, which is commonly sold in compressed air marketed for computer keyboard cleaning. The device uses the expansion of a portion of the cooling liquid into gas form as the liquid travels through a tube in order to absorb energy and cool the adjacent environment.

In an embodiment, a personal portable cooling system, includes:
a receiver including a molded entry port adapted to receive and secure a flow of coolant from a coolant container;
a tubing system including an entry port, a tubing loop, a tubing exhaust port and a plurality of temperature sensors in fluid communication with the receiver;
an entry valve disposed in the tubing entry port;
an exit valve disposed in the tubing exhaust port; and
a regulator, including a computer in communication with the entry valve, exit valve and the plurality of temperature sensors which controls the distribution of the coolant in the tubing system.

The system differs from modern refrigeration in that there is no compression cycle to force the gas back in liquid form or release heat into the nearby environment. Rather than relying on a compression cycle to pump another round of liquid around the loop, expanding into gas and removing more heat, the present device jettisons the liquid (as vapor) and replaces it with a new round of liquid from the can of compressed liquid.

A suitable source of cooling liquid includes a can of pressurized gas, such as an air duster. When the can is held upside down so that the liquid instead of the gas is loaded into the discharge nozzle, ice cold liquid is discharged when the trigger is depressed. Suitable liquids include diflouroethane or tetraflouroethane, such as R134a (also known as 1,1,1,2-tetraflouroethane).

In an embodiment, the system includes a wearable, portable refrigeration unit that is formed in the shape of a sleeve, a vest, shoulder pad, knee pad, or an ankle pad. The inner workings of this device would be very similar to that of a modern refrigerator, but the ‘compression cycle’ which is responsible for converting expanded gas refrigerant and compressing it back into liquid refrigerant would be completely absent. This is the most energy consuming step of the modern refrigerator, and requires the most hardware. By removing this step, one can shrink the benefits of immediate cooling into almost any common wearable item. The compressed gas is very inexpensive and small enough to be carried in a purse or handbag for use if one encounters swelling and pain while out and about or if there is an acute trauma that requires on site, rapid cooling to prevent excessive swelling and pain.

The device is manufactured in such a way that it locks a compressed air can (preferably one of the top 5 brands) into a position that places the liquid closest to the nozzle, with the gas resting above the liquid inside the can. Essentially, the can will be inverted, and placed into a regulator (a main component of the device).

The system components include a sleeve, regulator, tubing, and exhaust.

In an embodiment, the sleeve has at least two layers. The outer layer helps with fixing it around the limb, chest, or joint. The inner layer is a thin material that protects the patient's skin from the extreme cold that is possible when this liquid is pumped through the system.

The regulator is designed to receive a canister, for example, of pressurized tetraflouroethane or diflouroethane. The device has air tight seals, valves, and connections that allow for compressed, cold refrigerant to pass from the regulator to the tubing. The tubing bends back and forth in the pattern that is common for refrigerating devices and returns to the regulator. A valve within the regulator stops the cold liquid from immediately passing into the exhaust. This allows the entire system of tubes to fill with cold liquid. The cold liquid will cool the patient's skin for 15-20 minutes. When the patient is ready, they activate the exhaust valve which will evacuate the contents of the tubing system while leaving the valve connected to the canister closed. This allows the user to fill the tubing system with more cold liquid when they choose to do so.

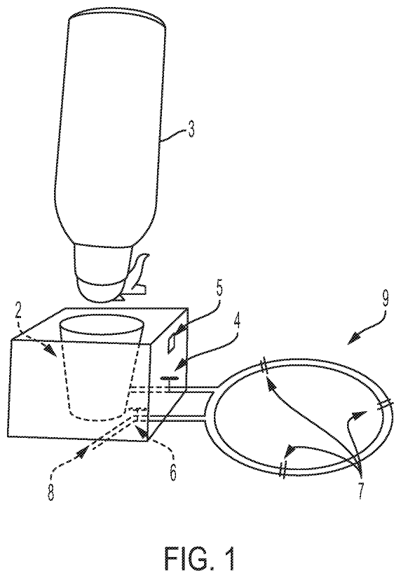

The tubing can be made of a material with the heat conduction of copper but with flexibility to provide for comfortable use once it is integrated into a sleeve or cuff. The material has a course that is similar to that of the standard refrigeration unit as shown in FIG. 1. The tube travels from the regulator along the entire area of the cuff, vest, blanket, etc. and then courses back to the regulator.

The exhaust can be a short tube or an open hole that allows the user to jettison the used refrigerant into the nearby environment. This clears the system of ‘warm’ liquid or gas and allows the user to introduce fresh cold liquid at the time of their choosing.

The regulator includes components: receiver—primarily composed of molded plastic which is the precise negative shape of the nozzle for a compressed air can. It will hold the air can in an inverted position, and will ensure that it does so in an airtight manner (utilizing rubber seals) so that freezing liquid can be introduced into the system without escaping. Each brand utilizes its own plastic shape for the nozzle/trigger assembly. This device will have receivers manufactured to perfectly match each of the 5 major brands. Computer: a computer will be responsible for controlling the distribution of liquid in the closed tube system that travels away from the regulator and back to it. Liquid traveling through the tube system is responsible for cooling the skin, joint, head, or chest of the patient.

Simply injecting compressed liquid through the tube system may typically not be enough to cool an injured limb or body part. Preferably, the liquid is injected into the tube system and is prevented from escaping the other end of the tube system in the form of a gas. The device is designed to work properly by introducing liquid into the closed system and the liquid is held at or near the pressure at which it is injected. In order to achieve cooling the valves are opened and closed in a very controlled, well-timed manner by the computer.

Additionally, the end of the tube system near the exhaust will have a tendency to trap room temperature air if the air is not permitted to be pushed out by the incoming, compressed liquid.

The computer controlled valves are opened initially as the compressed liquid is introduced. Once the system has been bled of room temperature air, the distal valve is closed, followed by closure of the near valve (at the entrance to the tube system). This will preserve the pressure of the cooled liquid, and will permit the tube system to cool skin, joints, extremities until the computer determines (by measuring the temperature at various points along the tube loop) that a round of compressed liquid is to be injected as the old liquid is purged through the exhaust. The computer can be programmed so that it can be set to maintain a preset temperature. It then goes on to trigger the liquid spray, close, and release valves in order to maintain that temperature until the patient's treatment is completed or the contents of the can have been exhausted.

The system delivers rapid and ice-cold therapy through wearable devices to various parts of the human body. The portable nature of the system provides an innovative and mobility-centered approach to situations where carrying ice, cold water, freeze packs or ice coolers is not practical or possible. The system delivers cold therapy to a variety of wearable devices such as a cooling vest, knee cryo-cuff, head wrap to treat acute head traumas, and other form fitting devices suitable to provide cold therapy. The system provides computer controlled release of compressed air canister to quickly cool wearable devices and is designed to eliminate the need to carry ice, cold water, and coolers. The system has universal adaptability with common compressed air canisters and can be worn on the body via shoulder strap or belt clip.

In an embodiment, a method for cooling a selected portion of a person with a portable cooling system, includes:
placing on a selected portion of a person a tubing system including an entry port, a tubing loop, a tubing exhaust port and a plurality of temperature sensors in fluid communication with a receiver;
securing a flow of coolant from a coolant container to the receiver;
closing an exit valve of the tubing exhaust port;
opening an entry valve of the tubing entry port;
controlling the distribution of the coolant in the tubing system with a regulator, including a computer in communication with the entry valve, exit valve and the plurality of temperature sensors. In an embodiment, the method further includes opening the exit valve to flush the system with the coolant present in the tubing system, closing the exit valve and opening the entry valve allowing coolant into the tubing system.

The disclosure will be further illustrated with reference to the attached Figs. It is understood that these Figs. are given by way of illustration and not meant to limit the disclosure or the claims to follow.

FIG. 1 shows a receiver 2 crafted from molded plastic in a shape that interfaces with a variety of commercially available can 3 of compressed air. The entry valve 4 closes under the control of the computer 5 to prevent back flow towards the spray nozzle. The exit valve 6 closes under the control of the computer to prevent expanding liquid and gas from leaving the system through the exhaust 8. The tubing 9 carries cooled liquid and is provided with temperature sensors 7 which communicate with the computer 5. The liquid cools as some of the diflouroethane expands from liquid into gas as it exits the spray nozzle and enters the tubing 9. The exhaust 8 is responsible for allowing expanded liquid (vapor) to pass into the nearby environment once the valve has been turned.

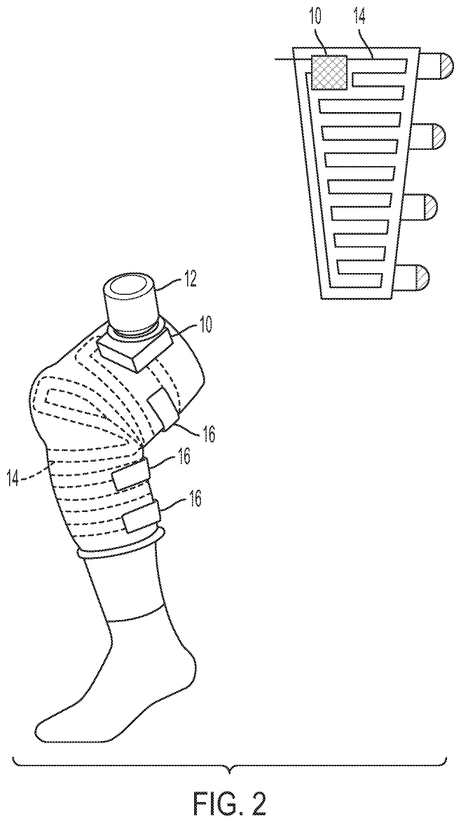

FIG. 2 shows a regulator 10 responsible for releasing pressurized, cold tetraflouroethane liquid from the canister 12. The regulator 10 provides the user with a read out of the pressure level in the tubing, temperature, and how much pressure remains in the canister. The cuff can include flexible, heat conducting tubes 14 shown as dotted lines between the layers of the cryo-cuff. A suitable material includes silicon. The cold tetraflouroethane is coursed through the tubing until it reaches a closed valve within the regulator. When the liquid in the tubes is no longer cool (it is warmed by the skin it is in contact with), the user can close the valve allows liquid to leave the canister 12, while opening the valve that leads to the outside atmosphere. This clears the system of warm gas, and allows the user to introduce fresh, cold liquid. The straps 16 are used to secure the cryo-cuff in place.

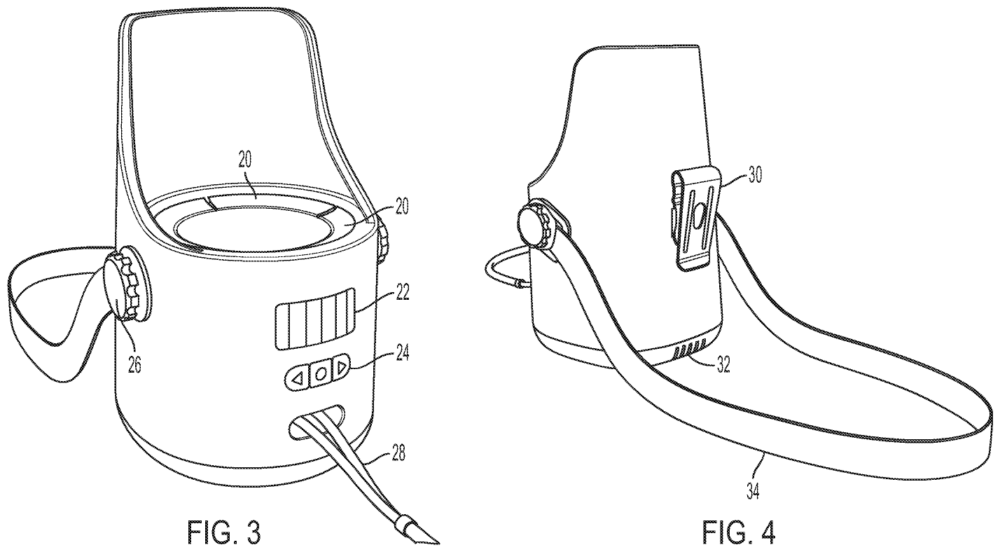

FIG. 3 shows rubber fins 20, LED screen 22, control interface 24, belt adjust knob 26 and cooling tube 28.

FIG. 4 shows a belt clip 30, exhaust vent 32 and removable shoulder strap 34.

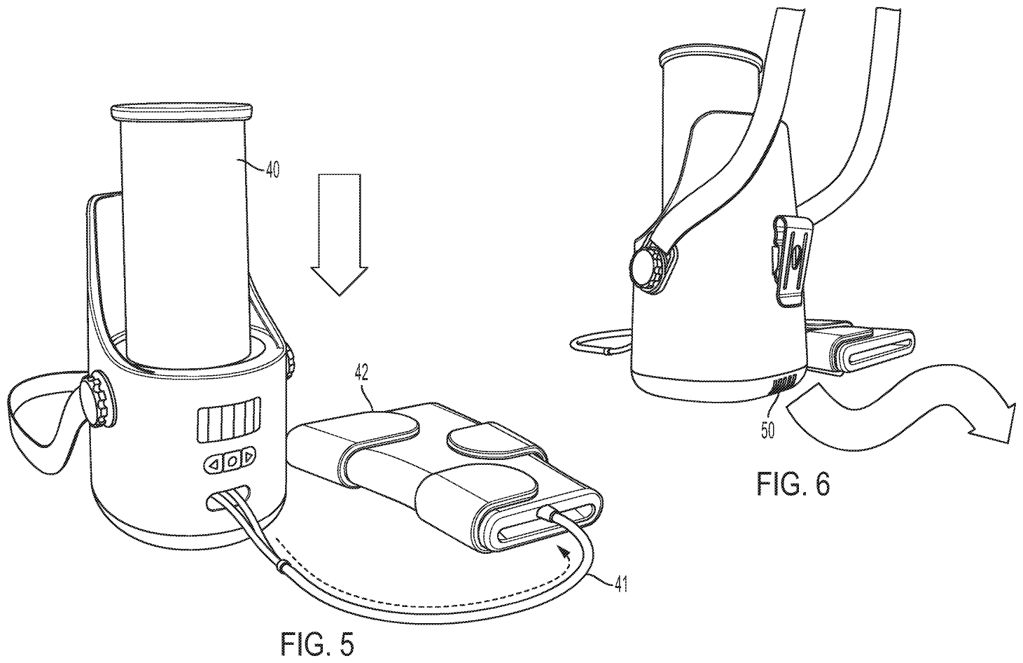

FIG. 5 shows the compressed air can 40 which plugs into the receiver and connected by tubing 41 to a knee pad 42.

FIG. 6 shows the exhaust vent 50 which allows expanded liquid to release from the system.

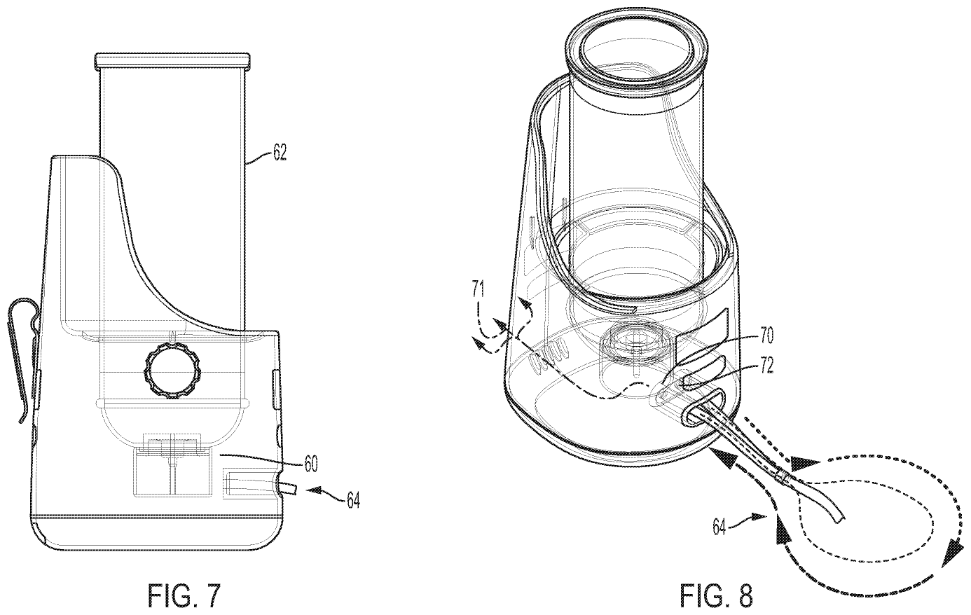

FIG. 7 shows a receiver 60 crafted from molded plastic to interface with available compressed air can 62. The tubing 64 carries cooled liquid. The liquid cools as diflouroethane expands from liquid into gas as it exits the spray nozzle and enters the tubing 64.

FIG. 8 shows the exit valve 70 which closes under the control of the computer to prevent expanding liquid and gas from leaving system through exhaust vent 71. The entry valve 72 closes under the control of the computer to prevent back flow towards the spray nozzle. The exhaust releases liquid (vapor) from device once release valve has been turned. The tubing 64 carries cooled liquid which cools as diflouroethane expands from liquid into gas as it exits the spray nozzle and enters the tubing 64.

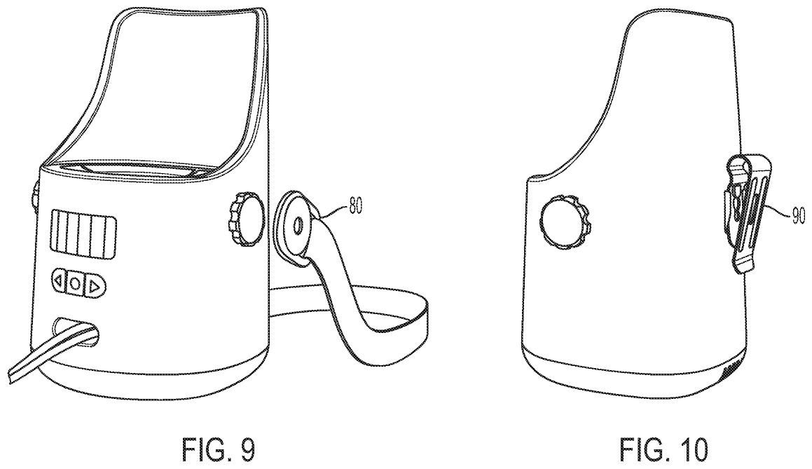

FIG. 9 shows unlock and remove belt 80.

FIG. 10 shows clip device 90 to belt or vest.

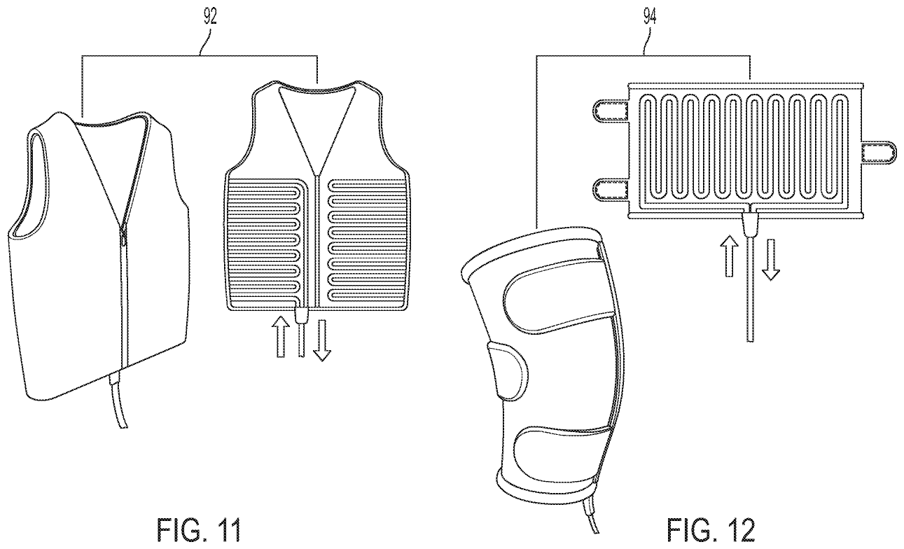

FIG. 11 shows a vest configuration 92.

FIG. 12 shows a knee pad configuration 94.

Although various embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

What is claimed:

1. A personal portable cooling system, comprising:
a receiver comprising a molded entry port adapted to receive and secure a flow of coolant from a coolant container;
a tubing system comprising an entry port, a tubing loop, a tubing exhaust port and a plurality of temperature sensors in fluid communication with the receiver;
an entry valve disposed in the tubing entry port;
an exit valve disposed in the tubing exhaust port; and
a regulator, comprising a computer in communication with the entry valve, exit valve and the plurality of temperature sensors which controls the distribution of the coolant in the tubing system.

2. The system of claim 1, wherein the coolant comprises compressed liquid.

3. The system of claim 2, wherein the compressed liquid comprises pressurized tetraflouroethane or diflouroethane.

4. The system of claim 1, wherein the coolant comprises a gas.

5. The system of claim 1, wherein the coolant comprises compressed gas.

6. The system of claim 1, wherein the coolant comprises CO₂.

7. The system of claim 1, wherein the tubing comprises a wearable device.

8. The system of claim 7, wherein the wearable device comprises a vest, head wrap, sleeve, shoulder pad, knee pad, or ankle pad.

9. A method for cooling a selected portion of a person with a portable cooling system, comprising:
placing on a selected portion of a person a tubing system comprising an entry port, a tubing loop, a tubing exhaust port and a plurality of temperature sensors in fluid communication with a receiver;
securing a flow of coolant from a coolant container to the receiver;
closing an exit valve of the tubing exhaust port;
opening an entry valve of the tubing entry port; and
controlling the distribution of the coolant in the tubing system with a regulator, including a computer in communication with the entry valve, exit valve and the plurality of temperature sensors.

10. The method according to claim 9, further comprising opening the exit valve to flush the system with the coolant present in the tubing system, closing the exit valve and opening the entry valve allowing coolant into the tubing system.

11. The method of claim 9, wherein the tubing system comprises a wearable device.

12. The method of claim 11, the wearable device comprises a vest, head wrap, sleeve, shoulder pad, knee pad, or ankle pad.
